(19) United States
(12) Patent Application Publication
(10) Pub. No.: US 2005/0026261 A1
(43) Pub. Date: Feb. 3, 2005

(54) Ethanol production by simultaneous saccharification and fermentation (SSF)

(75) Inventors: Eric Otto, Louisburg, NC (US); Jose Escovar-Kousen, Wake Forest, NC (US)

Correspondence Address: NOVOZYMES NORTH AMERICA, INC., 500 FIFTH AVENUE, SUITE 1600, NEW YORK, NY, 10110, US

(73) Assignee: Novozymes North America, Inc., Franklinton, NC (US)

(21) Appl. No.: 10/713,182

(22) Filed: Nov. 14, 2003

Related U.S. Application Data
(60) Provisional application No. 60/426,515, filed on Nov. 15, 2002.

(51) Int. Cl. C12P 7/06

(52) U.S. Cl. 435/161

(57) ABSTRACT

The invention relates to a process of producing ethanol by fermentation, said process comprising a simultaneous saccharification and fermentation (SSF) step conducted at a temperature of above 34° C. in the presence of a glucoamylase and a thermo-tolerant yeast.

### CROSS-REFERENCE TO RELATED APPLICATIONS

This application claims priority under 35 U.S.C. 119 of U.S. provisional application Ser. No. 60/426,515 filed on Nov. 15, 2002, the contents of which are fully incorporated herein by reference.

### BACKGROUND OF THE INVENTION

1. Field of the Invention

The invention relates to a process for producing ethanol comprising a simultaneous saccharification and fermentation step.

2. State of the Art

Ethanol has widespread application as an industrial chemical, gasoline additive or straight liquid fuel. As a fuel or fuel additive, ethanol dramatically reduces air emissions while improving engine performance. As a renewable fuel, ethanol reduces national dependence on finite and largely foreign fossil fuel sources while decreasing the net accumulation of carbon dioxide in the atmosphere. Fermentation processes are used for the production of ethanol. There are a large number of disclosures concerning production of alcohol by fermentation, among which are, e.g., U.S. Pat. No. 5,231,017 and CA 1,143,677. EP 138428 mentions an Aspergillus niger alpha-amylase preparation for use in liquefaction in the alcohol industry, and in WO 02/038787 a thermostable alpha-amylase preparation is suggested for this use. WO 99/28448 mentions the use of a thermostable glucoamylase in saccharification, and in simultaneous saccharification and fermentation (SSF) in a starch conversion process e.g. in the alcohol industry.

Due to the significant commercial interest in fuel ethanol, there is a continued need for further improvement of ethanol manufacturing processes, where even slight improvements have commercial significance.

### SUMMARY OF THE INVENTION

A problem to be solved by the present invention is how to reduce the amount of cooling required in an ethanol production process, while maintaining or even increasing ethanol yield or productivity, thus improving the cost-efficiency of the process. The invention relates to a process of producing ethanol by fermentation, said process comprising a simultaneous saccharification and fermentation (SSF) step conducted at a temperature of above 34° C. in the presence of a glucoamylase and a thermo-tolerant yeast. The elevated fermentation and/or saccharification temperature means that less cooling is required after the initial liquefaction step(s) which is(are) normally carried out at much higher temperatures.

The process of the invention may also comprise one or more additional steps, such as a recovery step of the produced ethanol.

The invention also relates to the products obtained or obtainable by the process of the invention, and to the use of such products, e.g. as fuel alcohol or an additive.

### BRIEF DESCRIPTION OF THE DRAWINGS

FIG. 1 illustrates a process flow diagram for the preparation of ethanol in accordance with one embodiment of the invention. The primary liquefaction step may be performed by the presence of the enzyme alpha-amylase in the slurry tank while the secondary liquefaction step is termed “liquefaction” on the diagram.

FIGS. 2, 3 and 4 show the results of three sets of SSF fermentation experiments on corn mash at three different temperatures 32° C., 35° C., and 37.5° C. Five different yeast strains were used. The following conditions were applied: 0.3 AFAU-units/g of AMG (Spirizyme™ Plus) combined with the five different yeast strains. Alcohol production was determined based on CO₂ weight loss (g) determination during the fermentation process and HPLC.

### DETAILED DESCRIPTION OF THE INVENTION

Ethanol production

Raw Material

In one embodiment, the starch containing material is selected from the group consisting of: tubers, roots and whole grain; and any combinations of the forgoing. In one embodiment, the starch containing material is obtained from cereals. The starch containing material may e.g. be selected from the groups consisting of corns, cobs, wheat, barley, cassava, sorghum, rye, milo and potatoes; or any combination of the foregoing.

In the ethanol processes of the invention, the starting raw material is preferably whole grain or at least mainly whole grain. A wide variety of starch containing whole grain crops may be used as raw material including: corn (maize), milo, potato, cassava, sorghum, wheat, and barley.

Thus, in one embodiment, the starch containing material is whole grain selected from the group consisting of corn (maize), milo, potato, cassava, sorghum, wheat, and barley; or any combinations thereof. In a preferred embodiment, the starch containing material is whole grain selected from the group consisting of corn, wheat and barley or any combinations thereof.

The raw material may also consist of or comprise a side stream from starch processing—e.g. C6 carbohydrate containing process streams that are not suited for production of syrups. In other embodiments, the raw material does not consist of or comprise a side stream from starch processing.

Process Steps

A process for producing ethanol by fermentation, said process comprising a simultaneous saccharification and fermentation (SSF) step conducted at a temperature of above 34° C. in the presence of a glucoamylase and a thermo-tolerant yeast.

In a preferred embodiment of the invention, the temperature is at least 34.5° C., or preferably at least 35° C.

The main process steps of the present invention may in one embodiment be described as separated into the following main process stages: milling (when whole grain is used as raw material), primary liquefaction, heat-treatment as provided by jet-cooking, secondary liquefaction, saccharification, fermentation, distillation.

The individual process steps of alcohol production may be performed batchwise or as a continuous flow. For the invention processes where all process steps are performed batch wise, or processes where all process steps are performed as a continuous flow, or processes where one or more process step(s) is(are) performed batch wise and one or more process step(s) is(are) performed as a continuous flow, are equally contemplated.

The cascade process is an example of a process where one or more process step(s) is(are) performed as a continuous flow and as such contemplated for the invention. For further information on the cascade process and other ethanol processes consult The Alcohol Textbook. Ethanol production by fermentation and distillation. Eds. T. P. Lyons, D. R. Kesall and J. E. Murtagh. Nottingham University Press 1995.

Milling

Thus, in a preferred embodiment of the process of the invention, the starch containing material is whole grain and the process comprises a step of milling the whole grain before step (a), i.e. before the primary liquefaction. In other words, the invention also encompasses processes of the invention, wherein the starch containing material is obtainable by a process comprising milling of whole grain, preferably dry milling, e.g. by hammer or roller mils. Grinding is also understood as milling.

In particular embodiments, the process of the invention further comprises prior to a primary liquefaction step, the steps of:
i. milling of whole grain;
ii. forming a slurry comprising the milled grain and water to obtain the starch containing material.

The whole grain is milled in order to open up the structure and allowing for further processing. Two processes of milling are normally used in alcohol production: wet and dry milling. The term “dry milling” denotes milling of the whole grain. In dry milling the whole kernel is milled and used in the remaining part of the process. Wet milling gives a good separation of germ and meal (starch granules and protein) and is with a few exceptions applied at locations where there is a parallel production of syrups.

Thus, in a preferred embodiment of the invention, dry milling is used since the secondary liquefaction step is advantageously included in dry milling processes for producing ethanol.

Liquefaction

In the liquefaction process the starch containing material, preferably in the form of milled whole grain raw material, is broken down (hydrolyzed) into maltodextrins (dextrins). In a preferred embodiment, in the primary liquefaction process of the invention the starch containing material, preferably in the form of milled whole grain raw material, is hydrolyzed to a DE (an abbreviation for dextrose equivalent) higher than 4. DE stands for “Dextrose equivalents” and is a measure of reducing ends on C6 carbohydrates. Pure glucose has DE of 100. Glucose (also called dextrose) is a reducing sugar. Whenever an amylase hydrolyzes a glucose-glucose bond in starch, two new glucose end-groups are exposed. At least one of these can act as a reducing sugar. Therefore the degree of hydrolysis can be measured as an increase in reducing sugars. The value obtained is compared to a standard curve based on pure glucose—hence the term dextrose equivalent. The DE may, e.g., be measured using Fehlings liquid by forming a copper complex with the starch using pure glucose as a reference, which subsequently is quantified through iodometric titration. In other words: DE (dextrose equivalent is defined as the amount of reducing carbohydrate (measured as dextrose-equivalents) in a sample expressed as w/w% of the total amount of dissolved dry matter. It may also be measured by the neocuproine assay (Dygert, Li Floridana(1965) Anal. Biochem. No 368). The principle of the neocuproine assay is that CuSO₄ is added to the sample, Cu²⁺ is reduced by the reducing sugar and the formed neocuproine complex is measured at 450 nm.

The hydrolysis may be carried out by acid treatment or enzymatically. The liquefaction is preferably carried out by enzymatic treatment, preferably an alpha-amylase treatment. In one embodiment, the liquefaction is carried out by preparing a slurry comprising milled raw material, preferably milled whole grain, and water, heating the slurry to between 60-95° C., preferably 80-85° C., and the enzyme(s) is (are) added to initiate liquefaction (thinning). This is also termed the “primary liquefaction”, i.e. it occurs before the process step of jet-cooking.

The liquefaction in the process of the invention is performed at any conditions (pH, temperature and time) found suitable for the liquefying enzyme used. Within the scope is a process of the invention, wherein a liquefaction step is performed at 60-95° C. for 10-120 min, preferably at 75-90° C. for 15-40 min. In one embodiment, the liquefaction step is performed at a pH in the range of about pH 4-7, preferably pH about 4.5-6.5. The pH of the slurry may by adjusted or not, depending on the properties of the enzyme(s) used. Thus, in one embodiment the pH is adjusted, e.g. about 1 unit upwards, e.g. by adding NH₃. The adjusting of pH is advantageously done at the time when the alpha-amylase is added. In a preferred embodiment, the pH is not adjusted and the alpha-amylase has a corresponding suitable pH-activity profile, such as being active at a pH about 4.

After the primary liquefaction step, the slurry is preferably jet-cooked at appropriate conditions to further gelatinize the starch, such as, e.g. at a temperature between 95-140° C., preferably 105-125° C. to ensure the gelanitization. In one embodiment, the jet-cooking step is performed under conditions 1-10 min, 105-150° C. and e.g. pH 4-7; preferably for 1-5 min, 105-120° C. and e.g. pH 4.5-6; such as, e.g., about 5 min, about 105° C., and e.g. pH about 5.0. As used herein, generally, the term jet-cooking also covers any other process which can be used to obtain a similar result.

Then the slurry is preferably cooled, e.g. to about 60-95° C. and more enzyme(s) is (are) added to obtain the final hydrolysis; the later is termed “secondary liquefaction”, i.e. liquefaction after jet-cooking which by the process of the invention is obtained by addition of at least a thermostable acid alpha-amylase or a thermostable maltogenic acid alpha-amylase.

The secondary liquefaction step is performed at suitable conditions (pH, temperature and process time). The secondary liquefaction may e.g., be performed at 60-95° C. for 10-120 min, preferably at 70-85° C. for 15-80 min and at pH 4.5-6.5. In one embodiment, the pH is not adjusted for the secondary liquefaction. In preferred embodiment, the pH during the secondary liquefaction is at most about 5.

In one preferred embodiment, in the secondary liquefaction step in the process of the invention the starch containing material, e.g. obtained from dry milled whole grain, is hydrolyzed to a DE in the range of about 5-15, e.g. 8-15, 8-14, such as, such as a DE in the range about 10-14., e.g. about 10-12.

The liquefaction process (both the primary and the secondary liquefaction process) is carried out at a suitable pH, e.g. at a pH in the range 4.5-6.5, such as at a pH between about 5 and about 6.

Milled and liquefied whole grain are also known as mash.

Saccharification

To produce low molecular sugars, DP₁₋₂, which can be metabolized by yeast, the maltodextrin from the liquefaction is preferably further hydrolyzed; this is also termed “saccharification”. The hydrolysis may be done enzymatically by the presence of a glucoamylase. An alpha-glucosidase and/or an acid alpha-amylase may also be present in addition to the glucoamylase.

A full saccharification step may last up to 72 hours. However, the saccharification and fermentation may be combined in simultaneous saccharification and fermentation (SSF) step, and in some embodiments of the invention a pre-saccharification step of 1-4 hours may be included. Pre-saccharification is carried out at any suitable process conditions. In a preferred embodiment, the pre-saccharification is carried out at temperatures from 30-65° C., such as around 60° C., and at, e.g., a pH in the range between 4-5, especially around pH 4.5.

Thus in one embodiment, the process of the invention may further comprise a pre-saccharification step, as described herein, which is performed after the secondary liquefaction step and before the SSF step.

In other embodiments, the process of the invention does not comprise a pre-saccharification and the saccharification is essentially only performed during fermentation, e.g. by the presence of a glucoamylase and optionally protease and/or phytase.

Fermentation

The microorganism used for the fermentation is added to the mash and the fermentation is ongoing until the desired amount of ethanol is produced; this may, e.g., be for 24-96 hours, such as 35-60 hours. The temperature and pH during fermentation is at a temperature and pH suitable for the microorganism in question, such as, e.g., in the range about 32-38° C., e.g. about 34° C., above 34° C., at least 34.5° C., or even at least 35° C., and at a pH e.g. in the range about pH 3-6, e.g. about pH 4-5.

In a preferred embodiment, a simultaneous saccharification and fermentation (SSF) process is employed where there is no holding stage for the saccharification, meaning that yeast and saccharification enzyme is added essentially together. In one embodiment, when doing SSF is introduced a pre-saccharification step at a temperature above 50° C., just prior to the fermentation.

In one embodiment, the fermentation is carried out in the presence of glucoamylase, protease, and/or phytase.

In a further embodiment, the addition of a thermostable acid alpha-amylase or a thermostable maltogenic acid alpha-amylase in the secondary liquefaction step in the process of the invention may make it possible to substitute the presence of glucoamylase activity in the fermentation step. Thus, one embodiment relates to a process of the invention for the production of ethanol, without addition of glucoamylase in the fermentation step or prior to the fermentation step.

Distillation

The process of the invention may further comprise recovering of the ethanol; hence the alcohol may be separated from the fermented material and purified. Following the fermentation the mash may be distilled to extract the ethanol. Ethanol with a purity of up to e.g. about 96 vol. % ethanol can be obtained by the process of the invention.

Thus, in one embodiment, the process of the invention further comprises a step of distillation to obtain the ethanol, wherein the SSF step and the distillation is carried out simultaneously or sequentially, optionally followed by one or more process steps for further refinement of the ethanol.

By-products from Distillation and Recycling:

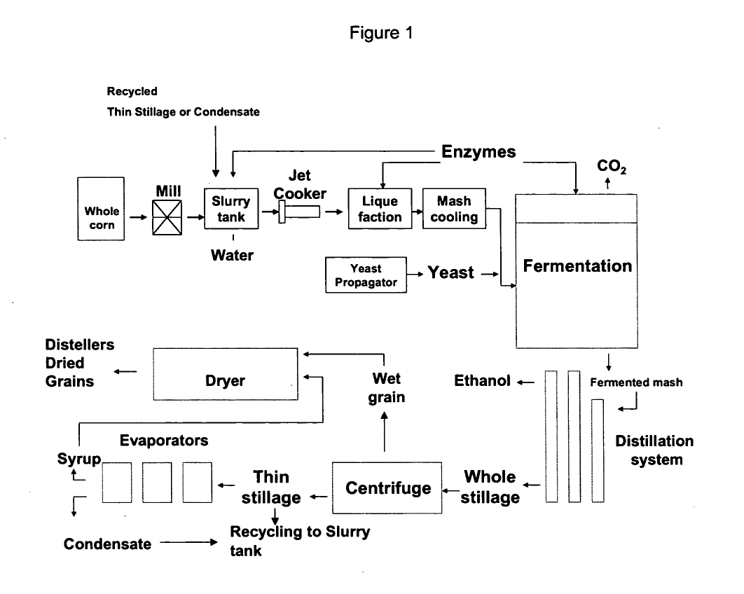

In one embodiment of the process of the invention, the aqueous by-product (“Whole Stillage”, cf. FIG. 1) from the distillation process is separated into two fractions, for instance by centrifugation: 1) “Wet Grain” (solid phase, see FIG. 1), and 2) “Thin Stillage” (Supernatant, see FIG. 1).

In one embodiment, in the process of the invention, the Thin Stillage (cf. FIG. 1) is recycled to the milled whole grain slurry.

The Wet Grain fraction may be dried, typically in a drum dryer. The dried product is referred to as “Distillers Dried Grains” (see FIG. 1), and can be used, e.g., as animal feed.

The Thin Stillage fraction may be evaporated providing two fractions (see FIG. 1):
1. a Condensate fraction of 4-6% DS (mainly of starch, proteins, and cell wall components), and
2. a Syrup fraction, mainly consisting of limit dextrins and non fermentable sugars, which may be introduced into a dryer together with the Wet Grains (from the Whole Stillage separation step) to provide a product referred to as “Distillers Dried Grain”, which also can be used as animal feed.

“Thin Stillage” is the term used for the supernatant of the centrifugation of the Whole Stillage (see FIG. 1). Typically, the Thin Stillage contains 4-6% DS (mainly starch and proteins) and has a temperature of about 60-90° C.

In another embodiment Thin Stillage is not recycled, but the condensate stream of evaporated Thin Stillage is recycled to the slurry containing the milled whole grain to be jet cooked.

Liquefaction Enzyme Activities

Alpha-amylase

The “primary liquefaction” is preferably performed in the presence of an alpha-amylase, e.g., derived from a micro-organism or a plant. Preferred alpha-amylases are of fungal or bacterial origin. Bacillus alpha-amylases (often referred to as “Termamyl-like alpha-amylases”), variant and hybrids thereof, are specifically contemplated according to the invention. Well-known Termamyl-like alpha-amylases include alpha-amylase derived from a strain of B. licheniformis (commercially available as Termamyl™), B. amyloliquefaciens, and B. stearothermophilus alpha-amylase. Other Termamyl-like alpha-amylases include alpha-amylase derived from a strain of the Bacillus sp. NCIB 12289, NCIB 12512, NCIB 12513 or DSM 9375, all of which are described in detail in WO 95/26397, and the alpha-amylase described by Tsukamoto et al., Biochemical and Biophysical Research Communications, 151 (1988), pp. 25-31. In the context of the present invention a Termamyl-like alpha-amylase is an alpha—amylase as defined in WO 99/19467 on page 3, line 18 to page 6, line 27. Contemplated variants and hybrids are described in WO 96/23874, WO 97/41213, and WO 99/19467, and include the Bacillus stearothermophilus alpha-amylase (BSG alpha-amylase) variant, alpha-amylase TTC, having the following mutations delta(181-182) +N193F (also denoted I181* +G182*+N193F) compared to the wild-type amino acid sequence set forth in SEQ ID NO: 3 disclosed in WO 99/19467. Contemplated alpha-amylase derived from a strain of Aspergillus includes Aspergillus oryzae and Aspergillus niger alpha-amylases.

Commercial alpha-amylase products and products containing alpha-amylases include TERMAMYL™ SC, FUNGAMYL™, LIQUOZYME™ SC and SAN™ SUPER, (Novozymes A/S, Denmark) and DEX-LO™, SPEZYME™ AA, and SPEZYME™ DELTA AA (from Genencor Int.).

Other contemplated alpha-amylase is the KSM-K36 alpha-amylase disclosed in EP 1,022,334 and deposited as FERM BP 6945, and the KSM-K38 alpha-amylases disclosed in EP 1,022,334, and deposited as FERM BP-6946.

The “secondary liquefaction” is performed in the presence of an alpha-amylase, in particular a thermostable acid alpha-amylase or a thermostable maltogenic acid alpha-amylase as described herein for use in the secondary liquefaction step in the process of the invention. The alpha-amylase is preferably derived from a micro-organism, including fungal and bacterial, or derived from a plant. Preferred thermostable acid alpha-amylases are of bacterial origin. Prefered thermostable maltogenic acid alpha-amylases are of fungal origin.

It is understood that enzymes are added in an effective amount for the actual conditions (temperature, pH) of the process, e.g. that the thermostable acid alpha-amylase is added in an amount effective in step (c).

In further embodiments of the process of the invention, in step (c) apart from the addition of the thermostable acid alpha-amylase is also added an alpha-amylase which is not a thermostable acid alpha-amylase.

The term “thermostable” in the context of a thermostable acid alpha-amylase means in one embodiment that the enzyme is active up to 90° C. at pH 5.0 using a 0.1 M citrate buffer and 4.3 mM Ca²⁺.

The thermostable acid alpha-amylase should have activity at the pH present during the liquefaction and fermentation, such as e.g. at a pH in the range pH 2.5-5.5 using a 0.1 M citrate buffer and 4.3 mM Ca²⁺. The enzyme should preferably at least be active in the range at pH 3-5. It is understood that the enzyme may also be active outside the pH ranges mentioned.

Examples of thermostable acid alpha-amylases as used herein are the alpha-amylase selected from the group consisting of LE399; the Aspergillus oryzae TAKA alpha-amylase (EP 238 023); the Aspergillus niger alpha-amylase disclosed in EP 383,779 B2 (section [0037] (see also the cloning of the A. niger gene in Example 1); the Aspergillus niger alpha-amylase disclosed in Example 1 of EP 140,410; Commercial fungal alpha-amylases FUNGAMYL® (Novozymes A/S); and Clarase™ (from Genencor Int., USA), the later both derived from Aspergillus.

By the expression “secondary liquefaction in the presence of a thermostable acid alpha-amylase” is understood liquefaction in the secondary liquefaction step in the process of the invention by treatment with an effective amount of a thermostable acid alpha-amylase” as defined herein.

The thermal/pH stability may be tested using, e.g., the following method: 950 micro liter 0.1 M Citrate+4.3 mM Ca²⁺ buffer is incubated for 1 hour at 60° C. 50 micro liter enzyme in buffer (4 AFAU/mI) is added. 2×40 micro liter samples are taken at 0 and 60 minutes and chilled on ice. The activity (AFAU/ml) measured before incubation (0 minutes) is used as reference (100%). The decline in percent is calculated as a function of the incubation time. To determine the Thermal stability the test is repeated using different temperatures, for instance 50, 60, 70, 80 and 90° C. To determine the pH stability the test is repeated using different pH's, for instance, pH 2.5; 3; 3.5; 4; 4.5; 5.

Other examples of alpha-amylases which may be used in the secondary liquefaction step in the process of the invention, is the alpha-amylase disclosed in Agric. Biol. Chem., 43:1165-1171,1979 by Guy-Jean Moulin and Pierre Galzy.

It is understood that the enzyme(s) is(are) added in an effective amount for the actual conditions (temperature, pH) of the process.

The thermostable maltogenic acid alpha-amylase should have activity at the pH present during the liquefaction and fermentation; such as e.g. at a pH in the range pH 2.5-5.5 using a 0.1 M citrate buffer and 4.3 mM Ca²⁺, a substrate consisting of DE 12 alpha-amylase TTC liquefied corn starch at 30% dry substance. The enzyme should preferably at least be active in the range at pH 3-5, preferably at least pH 2.5-5. It is understood that the enzyme may also be active outside the pH ranges mentioned.

The term “maltogenic” in the context of the invention, means that the enzyme is capable of releasing a relatively high amount of α-maltose as a product of its enzymatic activity.

In a particular interesting embodiment, the term “maltogenic” means that the enzyme using a DE 12 alpha-amylase TTC liquefied corn starch at 30% dry substance at 60° C., pH 4.5 and dosing the enzyme at 1 AFAU/g dry substance, the enzyme will in 24 hours catalyze the formation of at least 15%, or at least 20%, at least 25%, at least 30 w/w maltose as based on the total amount of starch. The maltose content may for instance be measured by HPLC as known by the person skilled in the art.

The term “DE 12 alpha-amylase TTC liquefied corn starch” in this context means that the substrate used for testing the maltogenicity of the alpha-amylase enzyme, is corn starch liquefied to a DE of 12 with alpha-amylase TTC.

The term “thermostable” means that the enzyme is relatively stable at higher temperatures. In one embodiment, the enzyme will maintain more than 90% of its activity for 1 hour at 70° C. using a DE 12 alpha-amylase TTC liquefied corn starch at 30% dry substance as substrate, pH 5.5, 0.1 M citrate buffer and 4.3 mM Ca²⁺.

The term “acid enzyme” means that the enzyme is relatively stable at low pH. In one embodiment, the enzyme will maintain more than 70% of its activity in the range from pH 3.5-5.0 (e.g. at pH 4), or preferably in the range from pH 3.8-4.7 (e.g. at pH 4.2) at the conditions: substrate DE 12 alpha-amylase TTC liquefied corn starch at 30% dry substance, Temperature 40° C., and 0.1 M citrate buffer and 4.3 mM Ca²⁺.

In one embodiment, the pH window (profile) of the enzyme used in the secondary liquefaction step in the process of the invention is as follows: the maximum activity of the enzyme is found at approximately pH 4.2 and/or the enzyme will maintain more than 70% of its activity in the range from pH 3.5-5.0 at the conditions: substrate is DE 12 alpha-amylase TTC liquefied corn starch at 30% dry substance, Temperature 40° C., and 0.1 M citrate buffer and 4.3 mM Ca²⁺.

In one embodiment, the temperature window (profile) of the alpha-amylase enzyme used in the secondary liquefaction step in the process of the invention is as follows: the enzyme will maintain more than 80% of its activity for 15 min in the range from 50-80° C. using a DE 12 alpha-amylase TTC liquefied corn starch at 30% dry substance as substrate. pH 5.5, 0.1 M citrate buffer and 4.3 mM Ca²⁺.

By the expression “secondary liquefaction in the presence of a thermostable maltogenic acid alpha-amylase” is understood liquefaction in the secondary liquefaction step by treatment with an effective amount of a thermostable maltogenic acid alpha-amylase” as defined herein. The alpha-amylase used in the secondary liquefaction is preferably a thermostable maltogenic acid alpha-amylase. The term “thermostable maltogenic acid” alpha-amylase”, means that the alpha-amylase is both thermostable, acid and maltogenic as defined herein. In one embodiment, the alpha-amylase is at least thermostable and acid as defined herein, optionally being maltogenic as defined herein.

The thermostable maltogenic acid alpha-amylase may be employed in the primary liquefaction step; however, the maximum effect is obtained if the enzyme is added the secondary liquefaction step.

Saccharification or SSF Enzyme Activities

Glucoamylase

The saccharification step or the simultaneous saccharification and fermentation (SSF) step may be carried out in the presence of a glucoamylase. The glucoamylase may be of any origin, e.g. derived from a microorganism or a plant. Preferred is glucoamylase of fungal or bacterial origin selected from the group consisting of Aspergillus niger glucoamylase, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Other contemplated Aspergillus glucoamylase variants include variants to enhance the thermal stability: G137A and G139A (Chen et al. (1996), Prot. Engng. 9, 499-505); D257E and D293E/Q (Chen et al. (1995), Prot. Engng. 8, 575-582); N182 (Chen et al. (1994), Biochem. J. 301, 275-281); disulphide bonds, A246C (Fierobe et al. (1996), Biochemistry, 35, 8698-8704; and introduction of Pro residues in position A435 and S436 (Li et al. (1997), Protein Engng. 10, 1199-1204. Furthermore, Clark Ford presented a paper on Oct. 17, 1997, ENZYME ENGINEERING 14, Beijing/China Oct 12-17, 97, Abstract number: Abstract book p.0-61. The abstract suggests mutations in positions G137A, N20C/A27C, and S30P in an Aspergillus awamori glucoamylase to improve the thermal stability. Other glucoamylases include Talaromyces glucoamylases, in particular derived from Talaromyces emersonii (WO 99/28448), Talaromyces leycettanus (U.S. Pat. No. Re. 32,153), Talaromyces duponti, Talaromyces thermopiles (U.S. Pat. No. 4,587,215). Bacterial glucoamylases contemplated include glucoamylases from the genus Clostridium, in particular C. thermoamylolyticum (EP 135,138), and C. thermohydrosulfuricum (WO 86/01831).

A preferred embodiment relates to the first aspect of the invention, wherein the glucoamylase is derived from Talaromyces emersonii, preferably from Talaromyces emersonii CBS 793.97; more preferably the glucoamylase has an amino acid sequence comprising one or more of the partial sequences shown in SEQ ID NOS: 1-6 of WO 99/28448; even more preferably the glucoamylase has an amino acid sequence with an identity of at least 60% with SEQ ID NO: 7 of WO 99/28448, or is a variant of the glucoamylase shown in SEQ ID NO: 7 of WO 99/28448.

Commercial products include SAN™ SUPER™; AMG™ E; and Spirizyme™ Plus (from Novozymes A/S).

Protease

In a preferred embodiment of the invention, the SSF step is carried out in the presence of a protease and/or a phytase.

Addition of protease(s) in the saccharification step, the SSF step and/or the fermentation step increase(s) the FAN (Free amino nitrogen) level and increase the rate of metabolism of the yeast and further gives higher fermentation efficiency.

Suitable proteases include microbial proteases, such as fungal and bacterial proteases. Preferred proteases are acidic proteases, i.e., proteases characterized by the ability to hydrolyze proteins under acidic conditions below pH 7.

In a preferred embodiment, the protease is selected from the group of fungal proteases, such as e.g. an acid fungal protease derived from a strain of Aspergillus.

Suitable acid fungal proteases include fungal proteases derived from Aspergillus, Mucor, Rhizopus, Candida, Coriolus, Endothia, Enthomophtra, Irpex, Penicillium, Sclerotiumand Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae, such as the pepA protease; and acidic proteases from Mucor pusillus or Mucor miehei.

Also contemplated are neutral or alkaline proteases, such as a protease derived from a strain of Bacillus. Bacterial proteases, which are not acidic proteases, include the commercially available products Alcalasee and Neutrase® (available from Novozymes A/S.

Additional Enzymes:

One or more additional enzymes may also be used during saccharification/pre-saccharification or SSF. Additional enzymes include e.g. pullulanase and/or phytase. Thus, in one embodiment, is added a glucoamylase and/or phytase in order to promote the fermentation.

Phytase:

In a preferred embodiment of the invention, the SSF step is carried out in the presence of a protease and/or a phytase.

The phytase used according to the invention may be any enzyme capable of effecting the liberation of inorganic phosphate from phytic acid (myo-inositol hexakisphosphate) or from any salt thereof (phytates). Phytases can be classified according to their specificity in the initial hydrolysis step, viz. according to which phosphate-ester group is hydrolyzed first. The phytase to be used in the invention may have any phytase specificity, e.g., be a 3-phytase (EC 3.1.3.8), a 6-phytase (EC 3.1.3.26) or a 5-phytase.

A suitable dosage of the phytase is e.g. in the range 5.000-250.000 FYT/g DS, particularly 10.000-100.000 FYT/g DS.

The phytase activity may be determined FYT units, one FYT being the amount of enzyme that liberates 1 micromole inorganic ortho-phosphate per min. under the following conditions: pH 5.5; temperature 37° C.; substrate: sodium phytate (C₆H₆O₂₄P₆Na₁₂) at a concentration of 0.0050 mole/I.

The phytase may be of any origin, such as, e.g. microbial, such as, e.g., derived from a strain of Peniophra lycii or Aspergillus oryzae. It may be produced recombinantly or non-recombinantly. The phytase may be derived e.g. from plants or microorganisms, such as bacteria or fungi, e.g., yeast or filamentous fungi.

The plant phytase may be from wheat-bran, maize, soy bean or lily pollen. Suitable plant phytases are described in Thomlinson et al, Biochemistry, 1 (1962), 166-171; Barrientos et al, Plant. Physiol., 106 (1994), 1489-1495; WO 98/05785; WO 98/20139.

A bacterial phytase may be from genus Bacillus, Pseudomonas or Escherichia, specifically the species B. subtilis or E. coli. Suitable bacterial phytases are described in Paver and Jagannathan, 1982, Journal of Bacteriology 151:1102-1108; Cosgrove, 1970, Australian Journal of Biological Sciences 23:1207-1220; Greiner et al, Arch. Biochem. Biophys., 303, 107-113, 1993; WO 98/06856; WO 97/33976; WO 97/48812.

A yeast phytase or myo-inositol monophosphatase may be derived from genus Saccharomyces or Schwanniomyces, specifically species Saccharomyces cerevisiae or Schwanniomyces occidentalis. The former enzyme has been described as a suitable yeast phytases are described in Nayini et al, 1984, Lebensmittel Wissenschaft und Technologie 17:24-26; Wodzinski et al, Adv. Appl. Microbiol., 42, 263-303; AU-A-24840/95.

Phytases from filamentous fungi may be derived from the fungal phylum of Ascomycota (ascomycetes) or the phylum Basidiomycota, e.g., the genus Aspergillus, Thermomyces (also called Humicola), Myceliophthora, Manascus, Penicillium, Peniophora, Agrocybe, Paxillus, or Trametes, specifically the species Aspergillus terreus, Aspergillus niger, Aspergillus niger var. awamori, Aspergillus ficuum, T lanuginosus (also known as H. lanuginosa), Myceliophthora thermophila, Peniophora lycii, Agrocybe pediades, Manascus anka, Paxillus involtus, or Trametes pubescens. Suitable fungal phytases are described in Yamada et al., 1986, Agric. Biol. Chem. 322:1275-1282; Piddington et al., 1993, Gene 133:55-62; EP 684,313; EP 0 420 358; EP 0 684 313; WO 98/28408; WO 98/28409; JP 7-67635; WO 98/44125; WO 97/38096; WO 98/13480.

Modified phytases or phytase variants are obtainable by methods known in the art, in particular by the methods disclosed in EP 897010; EP 897985; WO 99/49022; WO 99/48330.

Microorganism Used for Fermentation

In a preferred embodiment of the invention, the thermo-tolerant yeast is a yeast which when fermenting at 35° C. maintains at least 90% of the ethanol yields and 90% of the ethanol productivity during the first 70 hours of fermentation, as compared to when fermenting at 32° C. under otherwise similar conditions. Preferably, the thermo-tolerant yeast is a yeast which when fermenting at 35° C. is capable of producing at least 15% V/V alcohol from a corn mash comprising 34.5% (w/v) solids. Even more preferably, the thermo-tolerant yeast is Red Star®/Lesaffre Ethanol Red (commercially available from Red Star®/Lesaffre, USA, Product no: 42138).

Use of the Products Produced by the Method of the Invention

The ethanol obtained by the process of the invention may be used as, e.g., fuel ethanol; drinking ethanol, i.e., potable neutral spirits, or industrial ethanol, including fuel additive.

### EXAMPLES

### Example 1

Temperature

We evaluated the ethanol productivity (g/l-h) and ethanol yield (gr. of ethanol produced/gr. of ethanol expected) on corn mash (34.5% W/V solids) for a SSF step process for different yeast strains at 3 different temperatures.

Three sets of SSF fermentation experiments were performed on corn mash at three different temperatures 32° C., 35° C. and 37.5° C., and based on the procedure stated in the Standard Operating Procedure (SOP) No.: ABF-SM-5169.02/04 (1) which is available from Novozymes A/S upon request, and which is included herein in its entirety by reference. Five different yeast strains were used as follows:
1) Fermiol Super HA yeast.
2) Alltech yeast.
3) Red Star® Ethanol Red yeast.
4) Gert Strand Turbo TT yeast.
5) Baker's yeast.

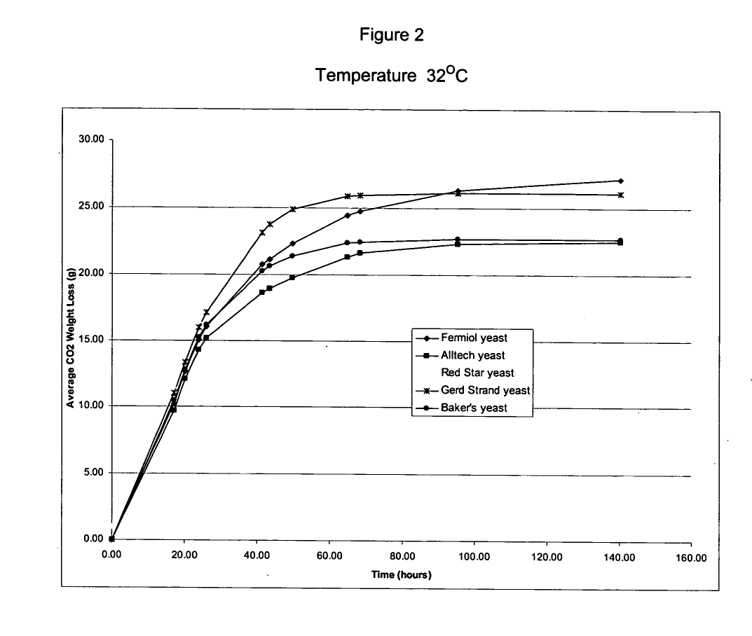

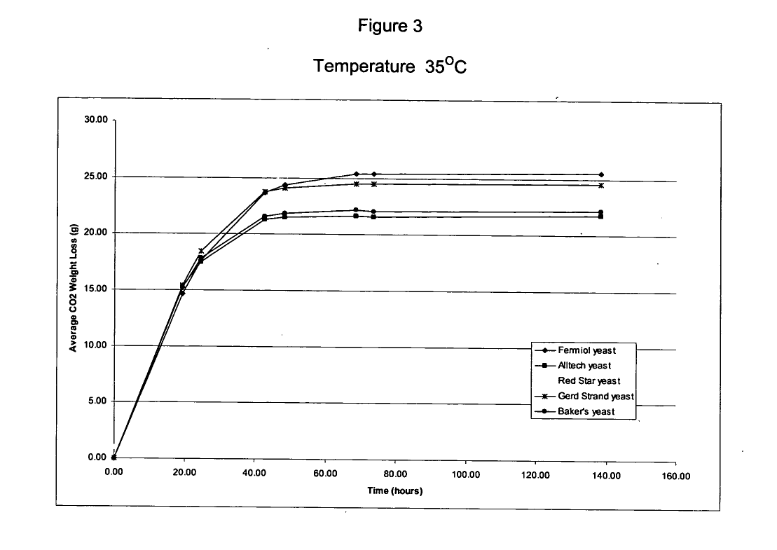

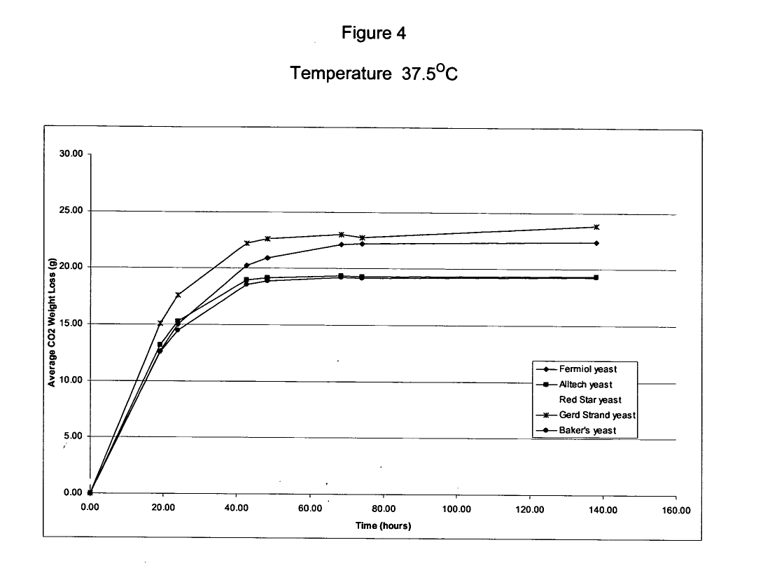

The following conditions were applied: 0.3 AFAU-units/g of AMG (Spirizyme Plus) combined with the five different yeast strains. Alcohol production was determined based on weight loss determination during the fermentation process and HPLC. Propagation was performed as established in the above-mentioned SOP. The results for the three sets of experiments can be seen in FIGS. 2, 3, and 4.

As expected, the temperature has an effect on the ethanol productivity and the yields of all yeasts, and the optimal temperature was 32° C. for all strains. An increase in temperature above 32° C. represents a higher residual glucose and lower ethanol yields (see Table 1).

TABLE 1

| | Residual | % Total | Ethanol | % Ethanol |
|---|---|---|---|---|
| | Sugar | Sugar | % V/V | Yield |
| 37.5° C. | | | | |
| Fermiol | 9.33 | 37.84 | 13.73 | 62.16 |
| Alltech | 11.19 | 45.36 | 11.78 | 54.64 |
| Red Star | 8.44 | 34.23 | 14.34 | 65.77 |
| Gert Strand | 8.75 | 35.47 | 14.11 | 64.53 |
| Baker's Yeast | 11.38 | 46.13 | 11.88 | 53.87 |
| 35° C. | | | | |
| Fermiol | 5.93 | 24.05 | 15.85 | 75.95 |
| Alltech | 9.54 | 38.68 | 13.48 | 61.32 |
| Red Star | 5.37 | 21.78 | 16.06 | 78.22 |
| Gert Strand | 6.88 | 27.89 | 15.08 | 72.11 |
| Baker's Yeast | 9.21 | 37.35 | 13.79 | 62.65 |
| 32° C.*** | | | | |
| Fermiol yeast | 4.85 | 19.65 | 17.03 | 80.35 |
| Alltech yeast | 9.61 | 38.95 | 13.93 | 61.05 |
| Red Star yeast | 4.23 | 17.15 | 17.10 | 82.85 |
| Gert Strand | 5.25 | 21.27 | 16.27 | 78.73 |
| Baker's yeast | 8.99 | 36.46 | 14.27 | 63.54 |

Notes to table 1:
*Residual sugar is DP1 + DP2 + DP3 + DP4⁺
**Ethanol Yield 2 is the theoretical ethanol yield based on residual sugar.
***Experiment at 32° C. was performed in the same corn mash but at different time

Higher temperatures are an additional stress factor for the yeast cells and increase cell mortality. The Red Star® Ethanol Red yeast was the number one performer in all cases. In general the strains' performance at different temperatures resemble the results at 32° C. and we continue observing a group a good performers: Red Star Ethanol Red, and Gert Strand and a group of poorer performers: Alltech Superstart and Baker's yeast and one strain in between: Fermiol.

1. A process for producing ethanol by fermentation, said process comprising a simultaneous saccharification and fermentation (SSF) step conducted at a temperature of above 34° C. in the presence of a glucoamylase and a thermo-tolerant yeast.

2. The process of claim 1, wherein the temperature is at least 34.5° C., or preferably at least 35° C.

3. The process of claim 1, wherein the glucoamylase is derived from Talaromyces emersonii, preferably from Talaromyces emersonii CBS 793.97.

4. The process of claim 1, wherein the glucoamylase has an amino acid sequence comprising one or more of the partial sequences shown in SEQ ID NOS: 1-6.

5. The process of claim 1, wherein the glucoamylase has an amino acid sequence with an identity of at least 60% with SEQ ID NO: 7, or is a variant of the glucoamylase shown in SEQ ID NO: 7.

6. The process of claim 1, further comprising recovery of the ethanol.

7. The process of claim 1, further comprising a step of distillation to obtain the ethanol, wherein the SSF step and the distillation is carried out simultaneously or sequentially.

8. The process of claim 1, wherein the thermo-tolerant yeast is a yeast which when fermenting at 35° C. maintains at least 90% of the ethanol yields and 90% of the ethanol productivity during the first 70 hours of fermentation, as compared to when fermenting at 32° C. under otherwise similar conditions.

9. The process of claim 1, wherein the thermo-tolerant yeast is a yeast which when fermenting at 35° C. is capable of producing at least 15% V/V alcohol from a corn mash comprising 34.5% (w/v) solids.

10. The process of claim 1, wherein the thermo-tolerant yeast is Red Star®/Lesaffre Ethanol Red (commercially available from Red Star®/Lesaffre, USA, Product no: 42138).

11. The process of claim 1, wherein the SSF step is carried out in the presence of a protease and/or a phytase.

12. The process of claim 11, wherein the protease is a microbial protease, preferably a fungal protease, more preferably an acid fungal protease, even more preferably an acid fungal protease derived from a strain of Aspergillus, preferably A. niger.

13. The process of claim 11, wherein the protease is a neutral or alkaline protease, such as a protease derived from a strain of Bacillus.

14. The process of claim 11, wherein the phytase is microbial, preferably the phytase is derived from a strain of Peniophra lycii or Aspergillus oryzae.

15. An ethanol produced by a process as defined in claim 1.


SEQUENCE LISTING (8 sequences)

SEQ ID NO: 1 - protein, 25 aa, Talaromyces emersonii
  misc_feature (13)..(13): Xaa can be any naturally occurring amino acid
ANGSLDSFLATEXPIALQGVLNNIG

SEQ ID NO: 2 - protein, 20 aa, Talaromyces emersonii
VQTISNPSGDLSTGGLGEPK

SEQ ID NO: 3 - protein, 22 aa, Talaromyces emersonii
  misc_feature (1)..(1): Xaa can be any naturally occurring amino acid
XNVNETAFTGPXGRPQRDGPAL

SEQ ID NO: 4 - protein, 35 aa, Talaromyces emersonii
DVNSILGSIHTFDPAGGCDDSTFQPCSARALANHK

SEQ ID NO: 5 - protein, 16 aa, Talaromyces emersonii
  misc_feature (2)..(2): Xaa can be any naturally occurring amino acid
TXAAAEQLYDAIYQWK

SEQ ID NO: 6 - protein, 35 aa, Talaromyces emersonii
AQTDGTIVWEDDPNRSYTVPAYCGQTTAILDDSWQ

SEQ ID NO: 7 - protein, 591 aa, Talaromyces emersonii
ATGSLDSFLATETPIALQGVLNNIGPNGADVAGASAGIVVASPSRSDPNYFYSWTRDAALTAKYLVDAFNRGNKDLEQTIQQYISAQAKVQTISNPSGDLSTGGLGEPKFNVNETAFTGPWGRPQRDGPALRATALIAYANYLIDNGEASTADEIIWPIVQNDLSYITQYWNSSTFDLWEEVEGSSFFTTAVQHRALVEGNALATRLNHTCSNCVSQAPQVLCFLQSYWTGSYVLANFGGSGRSGKDVNSILGSIHTFDPAGGCDDSTFQPCSARALANHKVVTDSFRSIYAINSGIAEGSAVAVGRYPEDVYQGGNPWYLATAAAAEQLYDAIYQWKKIGSISITDVSLPFFQDIYPSAAVGTYNSGSTTFNDIISAVQTYGDGYLSIVEKYTPSDGSLTEQFSRTDGTPLSASALTWSYASLLTASARRQSVVPASWGESSASSVLAVCSATSATGPYSTATNTVWPSSGSGSSTTTSSAPCTTPTSVAVTFDEIVSTSYGETIYLAGSIPELGNWSTASAIPLRADAYTNSNPLWYVTVNLPPGTSFEYKFFKNQTDGTIVWEDDPNRSYTVPAYCGQTTAILDDSWQ

SEQ ID NO: 8 - protein, 514 aa, B. stearothermophilus
AAPFNGTMMQYFEWYLPDDGTLWTKVANEANNLSSLGITALWLPPAYKGTSRSDVGYGVYDLYDLGEFNQKGAVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGIGKAWDWEVDTENGNYDYLMYADLDMDHPEVVTELKSWGKWYVNTTNIDGFRLDAVKHIKFSFFPDWLSDVRSQTGKPLFTVGEYWSYDINKLHNYIMKTNGTMSLFDAPLHNKFYTASKSGGTFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIAWSITTRPWTDEFVRWTEPRLVAW